(19) United States
(12) Patent Application Publication
(10) Pub. No.: US 2020/0215290 A1
(43) Pub. Date: Jul. 9, 2020

(54) RETRACTABLE OXYGEN TUBING AND SENSOR SYSTEM

(71) Applicant: Angela Marie Bell, Lebanon, CT (US)

(72) Inventor: Angela Marie Bell, Lebanon, CT (US)

(21) Appl. No.: 16/739,025

(22) Filed: Jan. 9, 2020

Related U.S. Application Data
(60) Provisional application No. 62/790,357, filed on Jan. 9, 2019.

(51) Int. Cl. A61M 16/10; G01V 9/00; B65H 75/44; B65H 75/40; A61M 16/06

(52) U.S. Cl. CPC A61M 16/1005; G01V 9/00; B65H 75/4486; A61M 25/002; A61M 16/0672; B65H 2701/33; B65H 75/403

(57) ABSTRACT

A retractable oxygen tubing and sensor system; the retractable oxygen tubing and sensor system includes a retractable oxygen tubing machine featuring a housing which conceals a supply of tubing hosted on a reel, and a motor, a wearable sensor and an internal or external machine sensor housed within the housing of the retractable oxygen tubing machine which is in communication with the wearable sensor and configured to control the motor to extend and alternatively retract the supply of tubing. The machine sensor is configured to sense proximity between the machine sensor and the wearable sensor. The retractable oxygen tubing and sensor system provides portable tangle free management of the supply of tubing to prevent falls of patients and tangling of tubing.

### CROSS-REFERENCE TO RELATED APPLICATION(S)

The present application is related to and claims priority to U.S. Provisional Patent Application No. 62/790,357 filed Jan. 9, 2019, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of oxygen tubing systems of existing art and more specifically relates to a retractable oxygen tubing system.

### RELATED ART

Patients that have continuous oxygen in home often spend an unnecessary amount of time and energy worrying about the positioning of their tubing. The tubing tends to get caught on objects, such as furniture, and patients have to continuously bend down to unsnag the tubing. In addition, the tubing becomes easily tangled when on the floor and can also be a trip hazard for the patient and others. A suitable solution is desired.

U.S. Pat. No. 2018/0289915 to Rhyan Dandre relates to a portable retractable reeling system and method for oxygen tubing management. The described portable retractable oxygen tubing reel provides improved portability and mechanical reeling functionality. The portable retractable oxygen tubing reel includes tubing wrapped around an inner circular component stored within an outer storage compartment whereby a handle attached to the inner circular component may be rotated to wind or unwind tubing from the portable retractable oxygen tubing reel. Tubing may then be connected to a medical oxygen storage device at one end and a nasal oxygen cannula positioned near in proximity to a patient's nose for breathing oxygen at the other end.

### SUMMARY OF THE INVENTION

In view of the foregoing disadvantages inherent in the known oxygen tubing system art, the present disclosure provides a novel retractable oxygen tubing and sensor system. The general purpose of the present disclosure, which will be described subsequently in greater detail, is to provide an effective and efficient retractable oxygen tubing and sensor system.

A retractable oxygen tubing and sensor system is disclosed herein. The retractable oxygen tubing and sensor system includes a retractable oxygen tubing machine including a housing which conceals a supply of tubing hosted on a reel, and a motor, a wearable sensor and an internal or external machine sensor housed within the housing of the retractable oxygen tubing machine which is in communication with the wearable sensor and configured to control the motor to extend and alternatively retract the supply of tubing. The machine sensor is configured to sense proximity between the machine sensor and the wearable sensor. The retractable oxygen tubing and sensor system provides portable tangle free management of the supply of tubing to prevent falls of patients and tangling of tubing.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a retractable oxygen tubing and sensor system, constructed and operative according to the teachings of the present disclosure.

FIG. 1 is a perspective view of the retractable oxygen tubing and sensor system during an ‘in-use’ condition, according to an embodiment of the disclosure.

FIG. 2 is a perspective view of the retractable oxygen tubing and sensor system of FIG. 1, according to an embodiment of the present disclosure.

FIG. 3 is a cut away view of the retractable oxygen tubing and sensor system of FIG. 1, according to an embodiment of the present disclosure.

FIG. 4 is a perspective view of the retractable oxygen tubing and sensor system of FIG. 1, according to an embodiment of the present disclosure.

FIG. 5 is a perspective view showing various embodiments of the retractable oxygen tubing and sensor system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to oxygen tubing systems and more particularly to a retractable oxygen tubing system as used to improve the use of oxygen delivering machines and tubing while reducing trip hazards and tangling of the tubing.

Generally, the retractable oxygen tubing and sensor system is for use with oxygen machines. The present invention provides a retractable oxygen tube system designed to keep tubing free of tangles and elevated from the floor. The device prevents falls and frustration experienced by patients that have continuous oxygen in the home. The device keeps the tubing at a comfortable length from the user. The present invention eliminates the need to continuously reposition tubing and spend time worrying about potential obstacles. The device will be connected to the machine or nearby. It can be attached to the machine, built into the machine or a separate device. The device has a sensor and a few hundred feet of oxygen tubing. The machine sensor tells the motor to move wheels forward or backward depending on the user's distance. The inside of the unit has a spool of tubing and a tube guide. The tubing is fed through two motor-powered wheels. The exact specifications may vary.

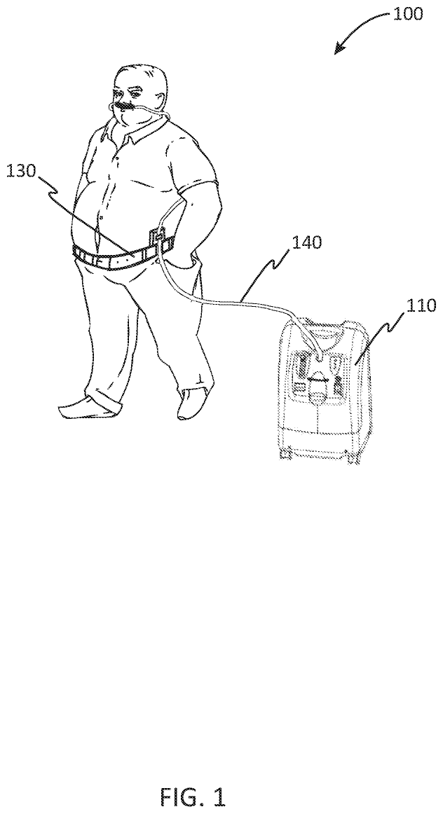

Referring now more specifically to the drawings by numerals of reference, there is shown in FIGS. 1-5, various views of a retractable oxygen tubing and sensor system 100.

FIG. 1 shows a retractable oxygen tubing and sensor system 100 during an ‘in-use’ condition, according to an embodiment of the present disclosure. Here, the retractable oxygen tubing and sensor system 100 may be beneficial for use by a user to provide portable tangle free management of a supply of oxygen tubing 140 to prevent falls of patients and tangling of oxygen tubing 140. As illustrated, the retractable oxygen tubing and sensor system 100 may include a portable retractable oxygen tubing machine 110 including a housing 112 which conceals a supply of tubing 140 hosted on a reel 114, and a motor 116, a wearable sensor 130 and an internal or external machine sensor 132 housed within the housing 112 of the retractable oxygen tubing machine 110 which is in communication with the wearable sensor 130 and configured to control the motor 116 to extend and alternatively retract the supply of tubing 140. The machine sensor 132 is configured to sense proximity between the machine sensor 132 and the wearable sensor 130.

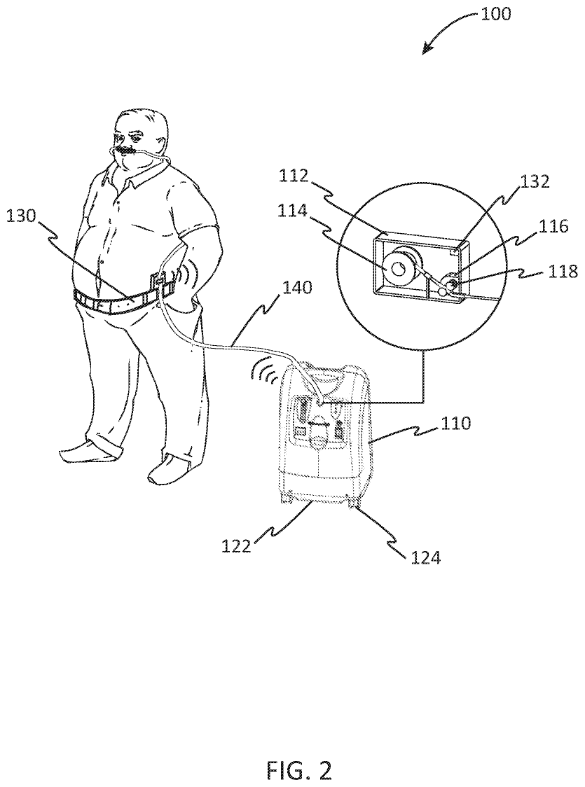

FIG. 2 shows the retractable oxygen tubing and sensor system 100 of FIG. 1, according to an embodiment of the present disclosure. As above, the retractable oxygen tubing and sensor system 100 may include the retractable oxygen tubing machine 110 including the housing 112 concealing the supply of tubing 140 hosted on the reel 114, the motor 116, two motor-powered wheels 118 powered by the motor 116 and configured to extend the supply of tubing 140 through an opening aperture 120 of the housing 112, and a tube guide 126 directing the supply of tubing 140 from the reel 114 towards the two motor-powered wheels 118 and the opening aperture 120. The supply of tubing 140 is provided to deliver oxygen to the user through the supply of tubing 140 and out through a nasal cannula. The system 100 further comprises an internal power source for powering the device. The supply of tubing 140 includes at least 100 feet of tubing. The retractable oxygen tubing and sensor system 100 is provided to keep tubing free of tangles and elevated from the floor. The device prevents falls and frustration experienced by patients that have continuous oxygen in the home.

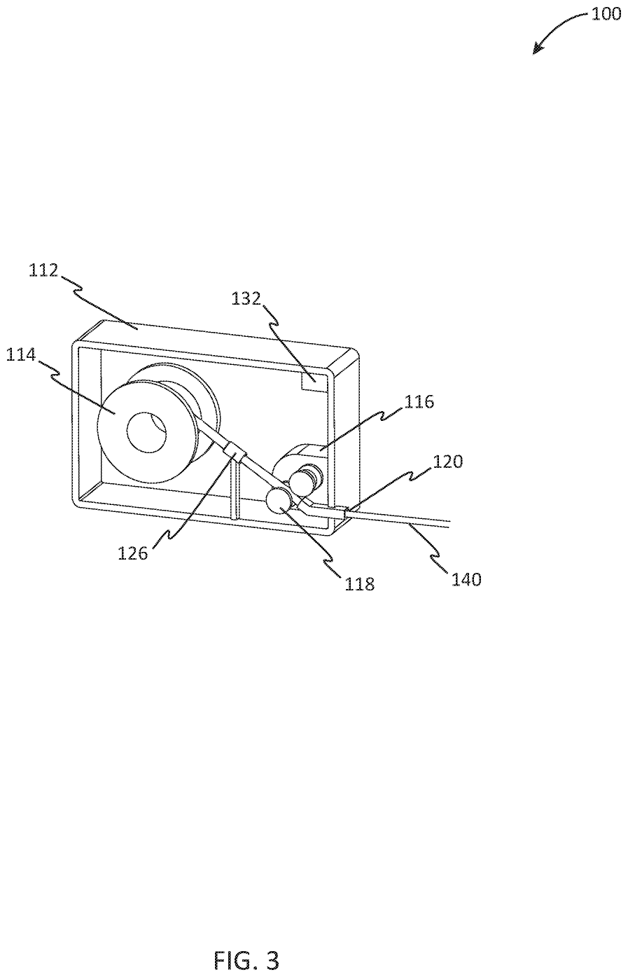

FIG. 3 is a cut away view of the retractable oxygen tubing and sensor system 100 of FIG. 1, according to an embodiment of the present disclosure. More specifically, the housing 112 of the retractable oxygen tubing machine 110 houses the supply of tubing 140, the reel 114, the motor 116, two motor-powered wheels 118, and the tube guide directing the supply of tubing 140 from the reel 114 towards the two motor-powered wheels 118 and the opening aperture 120. The retractable oxygen tubing machine 110 includes four external wheels 124 positioned on a base 122 of the housing 112. The retractable oxygen tubing machine 110 including the machine sensor 132 which activates the motor 116 to move the external wheels 124 in order to follow the user wearing the wearable sensor 130 and stay at an appropriate distance as the machine sensor 132 senses proximity between the machine sensor 132 and the wearable sensor 130. The reel 114 is configured to rotate during extending and retracting of the supply of tubing 140.

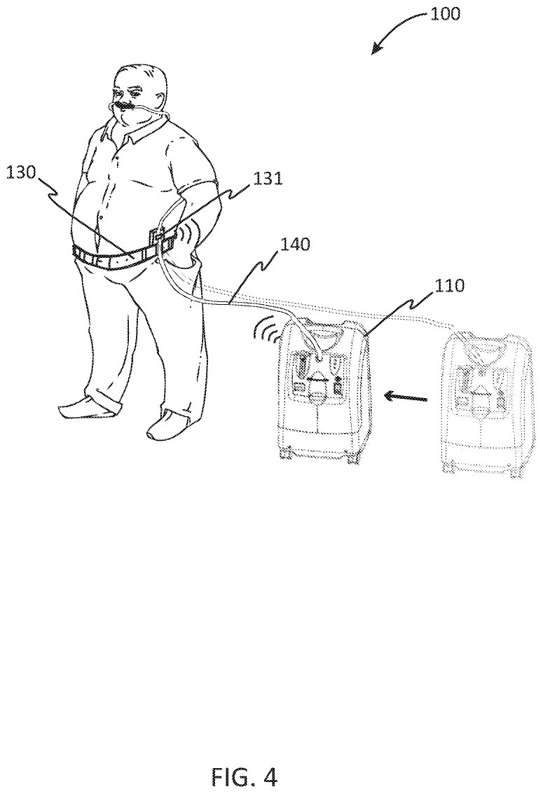

FIG. 4 is a perspective view of the retractable oxygen tubing and sensor system 100 of FIG. 1, according to an embodiment of the present disclosure. The wearable sensor 130 and the machine sensor 132 housed within the housing of 112 the retractable oxygen tubing machine 110 are in communication and configured to control the motor 116 to extend and alternatively retract the supply of tubing 140. As shown in the FIGS. the wearable sensor 130 is provided in a waist belt configuration. The wearable sensor 130 comprises an attachment point 131 configured to receive and support a portion of the supply of tubing 140. The retractable oxygen tubing machine 110 keeps the supply of tubing 140 at an elevated position above a floor surface to prevent tripping and tangling of the supply or tubing 140.

According to one embodiment, the retractable oxygen tubing and sensor system 100 may be arranged as a kit including the wearable sensor 130 and the machine sensor 132 for use with an oxygen tubing machine 110. In particular, the retractable oxygen tubing and sensor system 100 may further include a set of instructions. The instructions may detail functional relationships in relation to the structure of the retractable oxygen tubing and sensor system 100 such that the retractable oxygen tubing and sensor system 100 can be used, maintained, or the like, in a preferred manner.

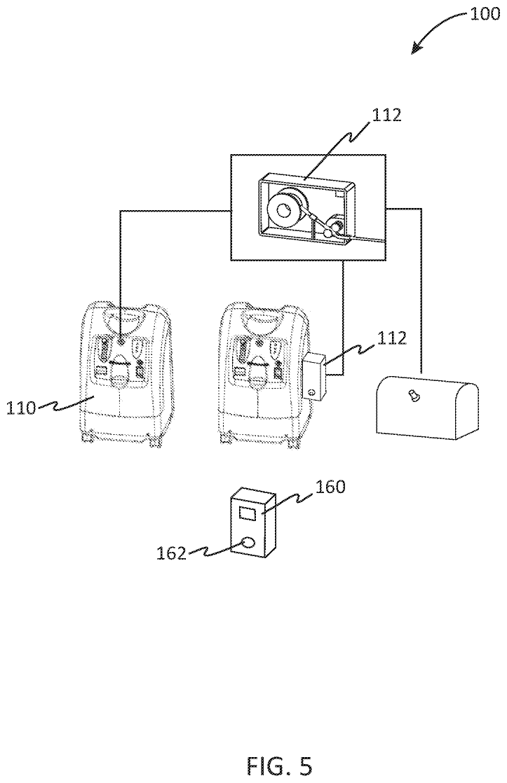

FIG. 5 is a perspective views of various embodiments of the retractable oxygen tubing and sensor system 100 of FIG. 1, according to an embodiment of the present disclosure. The machine sensor 132 of the present invention may be an external, non-attached device in communication with the retractable oxygen tubing machine 110. Alternatively, the machine sensor 132 may be an external attachment to the retractable oxygen tubing machine 110. In certain embodiments, the system further includes a handheld device 160 configured to remotely control extending and retracting functions of the supply of tubing 140. The handheld device 160 is in wireless communication with the machine sensor 132 allowing for remote controlling of the system. The handheld device 160 includes a push button 162 configured to activate retraction of the supply of tubing 140 when pushed.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A retractable oxygen tubing and sensor system comprising:
a retractable oxygen tubing machine including
a housing comprising
tubing wound on a reel,
and
a motor operably connected to the reel;
a machine sensor operably connected to the motor;
and
a wearable sensor,
wherein the machine sensor senses proximity between the wearable sensor and the machine sensor and controls the motor to extend or retract the tubing a length proportional to the proximity in order to provide tubing management to alleviate tangling and reduce patient falls.

2. The retractable oxygen tubing and sensor system of claim 1, wherein the housing further comprises two motor-powered wheels powered by the motor or a second motor and configured to extend or retract the tubing through an opening in the housing.

3. The retractable oxygen tubing and sensor system of claim 2, wherein the housing further comprises a tube guide disposed between the reel and the two motor-powered wheels.

4. The retractable oxygen tubing and sensor system of claim 1, wherein the tubing is configured to connect to a nasal cannula of the patient.

5. The retractable oxygen tubing and sensor system of claim 1, wherein the retractable oxygen tubing machine is portable.

6. The retractable oxygen tubing and sensor system of claim 5, wherein the retractable oxygen tubing machine includes wheels operable to support the machine positioned in or on a base of the housing.

7. The retractable oxygen tubing and sensor system of claim 1, wherein the reel rotates to extend or retract the tubing.

8. The retractable oxygen tubing and sensor system of claim 1, wherein the retractable oxygen tubing machine holds the tubing above a floor to alleviate tripping and tangling of the tubing.

9. The retractable oxygen tubing and sensor system of claim 1, wherein the wearable sensor is in a waist belt.

10. The retractable oxygen tubing and sensor system of claim 9, wherein the wearable sensor comprises a tubing attachment point configured to receive and support a portion of the tubing.

11. The retractable oxygen tubing and sensor system of claim 1, wherein the machine sensor is configured to activate the motor or another motor to propel the machine towards the wearable sensor proportional to the proximity.

12. The retractable oxygen tubing and sensor system of claim 1, wherein the machine sensor is an external sensor.

13. The retractable oxygen tubing and sensor system of claim 12, wherein the machine sensor is not attached to, but is in communication with, the retractable oxygen tubing machine.

14. The retractable oxygen tubing and sensor system of claim 1, wherein the system further includes a handheld device configured to remotely extend or retract the supply of tubing.

15. The retractable oxygen tubing and sensor system of claim 14, wherein the handheld device includes a button configured to retract the tubing when pushed.

16. The retractable oxygen tubing and sensor system of claim 15, wherein the handheld device is in wireless communication with the machine sensor.

17. The retractable oxygen tubing and sensor system of claim 1, wherein the tubing is at least 100 feet long.

18. The retractable oxygen tubing and sensor system of claim 1, wherein the system further comprises an internal power source.

19. A retractable oxygen tubing and sensor system, the retractable oxygen tubing and sensor system comprising:
a portable, retractable oxygen tubing machine including
a housing comprising
tubing wound on a reel,
a motor operably connected to the reel,
an opening in the housing,
two motor-powered wheels powered by the motor or a second motor and configured to extend or retract the tubing through the opening,
a base,
wheels operable to support the machine positioned in or on the base,
and
a tube guide disposed between the reel and the wheels;
a machine sensor in communication with the motor;
and
a wearable waist-belt sensor that comprises a tubing attachment point configured to receive and support a portion of the tubing,
wherein the tubing is configured to attach to a nasal cannula of a patient,
wherein the reel rotates to extend or retract the tubing,
wherein the retractable oxygen tubing machine holds the tubing above a floor to alleviate tripping and tangling of the tubing,
wherein the machine sensor senses proximity between the wearable sensor and the machine sensor and controls the motor to extend or retract the tubing a length proportional to the proximity to provide tubing management to alleviate tangling and reduce patient falls, and
wherein the machine sensor is configured to activate the motor or another motor to propel the machine towards the wearable sensor a distance proportional to the proximity.

20. The retractable oxygen tubing and sensor system of claim 19, further comprising a set of instructions; and wherein the retractable oxygen tubing and sensor system is arranged as a kit.
